(19) United States
(12) Patent Application Publication
(10) Pub. No.: US 2003/0135118 A1
(43) Pub. Date: Jul. 17, 2003

(54) Apparatus for in vivo monitoring of the effect of antiangiogenic drugs on cancers

(76) Inventor: David J Watmough, Inverness (GB)

Correspondence Address: John L Cordani, Carmody & Torrance, P O Box 1110, 50 Leavenworth Street, Waterbury, CT, 06721-1110, US

(21) Appl. No.: 10/203,368

(22) PCT Filed: Feb. 9, 2001
(86) PCT No.: PCT/GB01/00549

(30) Foreign Application Priority Data
Feb. 11, 2000 (GB) 0003032.0

(51) Int. Cl. A61B 8/02

(52) U.S. Cl. 600/454

(57) ABSTRACT

The invention here described relates to monitoring the effect of angiogenesis inhibiting drugs used to treat cancer in breast and testicular tissues. A probe combining electromagnetic radiation in the red and infra-red part of the spectrum is physically combined with a series of Ultrasound Doppler transducers arranged to monitor blood flow over one hemisphere of the tumour. This arrangement overcomes the major difficulty with conventional Doppler blood flow probes that the signals vary more from area to area of the cancer than the changes induced by the drugs acting on the neovascularisation over short time intervals. The invention envisages using probes with transducers of different frequencies and where the piezoelements are angled to cover such an area of angiogenesis that inhomogeneities become insignificant, by virtue of the fact that signals from all the vessels in a hemispherical volume of the tumour's advancing front are collected and processed electronically to produce the observed spectra.

The use of an instrument combining light and Doppler Ultrasound to detect breast cancer [U.S. Pat. No. 5,007,428 Watmough] has been previously described. A preliminary study using the device (Brittenden J. Watmough D. J. Heys S. D. and Eremin O. [1995] Preliminary clinical evaluation of a combined optical Doppler ultrasound instrument for the detection of breast cancer. Brit. J. Radiol. 68, 1344-1348) on a series of patients with a variety of breast conditions demonstrated that 73% of cancers could be detected. It was not initially appreciated that the camera, tumour and light source should be placed on a common axis to achieve optimum sensitivity. Therefore it is expected that in future studies where this requirement is achieved from the outset that higher detection rates will be achievable. The optical examination reveals the presence of the cancer by virtue of excess absorption due to the presence of angiogenesis [i.e. blood vessels induced by the cancer to supply it with additional oxygen and nutrients for more rapid growth] around the periphery of the tumour. However haematoma and bruising can give rise to images which can be mistaken for cancers. The ultrasound Doppler examination was incorporated to exclude these latter conditions by virtue of the fact that no blood flow is associated with them. In such cases no Doppler frequency shift signals can be elicited from the periphery of the lesion. Most of the cancers which went undetected by the optical and Doppler ultrasound based instrument were small and had not developed neovascularisation or were demonstrated on X-ray solely by the presence of microcalcification. The important point to note here is that the optical/Doppler instrument worked well on virtually all larger advanced cancers.

Recently the idea of treating advanced cancers with angiogenesis inhibiting drugs has been proposed with a view to starving the cancer of oxygen and nutrients and therefore preventing further growth and hopefully shrinking it. The proposed new method of treatment has another potential advantage namely that any secondary tumours should be treated simultaneously.

There is therefore a requirement for an instrument to monitor the cancer at intervals over a protracted period and to reveal the extent of neovascularisation and changes in it. A modified version of the optical / ultrasound Doppler instrument described in U.S. Pat. No. 5,007,428 is ideal for this purpose. The present patent application describes an instrument capable of producing images of cancerous tumours and Doppler ultrasound frequency shift spectra, deriving from blood flow around them, which would be identical on successive occasions unless the antiangiogenesis drugs were having an effect on the extent of neovascularisation.

In the earlier study it was found that the angiogenesis around a cancer is not homogeneous and that using a small conventional Doppler probe with two typically millimetre-sized elements fails to produce Doppler shift spectra which can be meaningfully compared. It is necessary to devise a means of replacing the Doppler transducers [transmitter and receiver] in the same position in relation to the cancer on successive occasions. The direction of the transducer axes must also be reproduced on each occasion. The present application describes an instrument by means of which serial examinations of cancers using light and Doppler ultrasound can reveal the efficacy of treatments. Serial examinations by light show the image of the cancer diminishing in area over time as a result of successful therapy. The Doppler frequency shift spectra show diminishing signal amplitude and reduced values of frequency shift maxima, corresponding to the shrinking and or disappearance of vessels around the tumour. Complete destruction of neovascularisation leads to disappearance of the associated ultrasound Doppler frequency shift signals.

On previous occasions when using a conventional Doppler probe to interrogate a cancer serially [e.g. weekly intervals], the signals could not be interpreted in terms of changes in angiogenesis because there was no means of being sure that the position of the probe was the same on each occasion.

The transcutaneous measurement of blood flow velocity by ultrasound Doppler frequency shift is a well-established technique, Wells et al [1977].

When an ultrasonic wave of frequency f and velocity c is reflected by blood flowing with velocity v, the Doppler shift f_{D} is given by:

f_{D}=fᵣ−f where fᵣ is the received frequency.

It can be shown that

f_{D}=−(2f·v·cos x)/c

where x is the angle between the direction of flow and the axis of the ultrasound beam, and c the velocity of ultrasound is much greater than v.

In soft tissues and in blood c=1500 m s⁻¹, and for f=8 MHz then f_{D}=2 kHz when v=100 mm s⁻¹ and cos x=1.

Transducers a few mm in diameter can be used to explore flow through a single vessel in a selective and sensitive fashion. In our study we examine signals generated by a chaotic mass of vessels (rather than a single one) and this is the reason for a spectrum of frequency shifts. Cancers give rise to high amplitude signals and high flow velocities (notably from arteriovenous shunts). Another characteristic of flow around tumours is positive (non-zero) flow throughout the cardiac cycle.

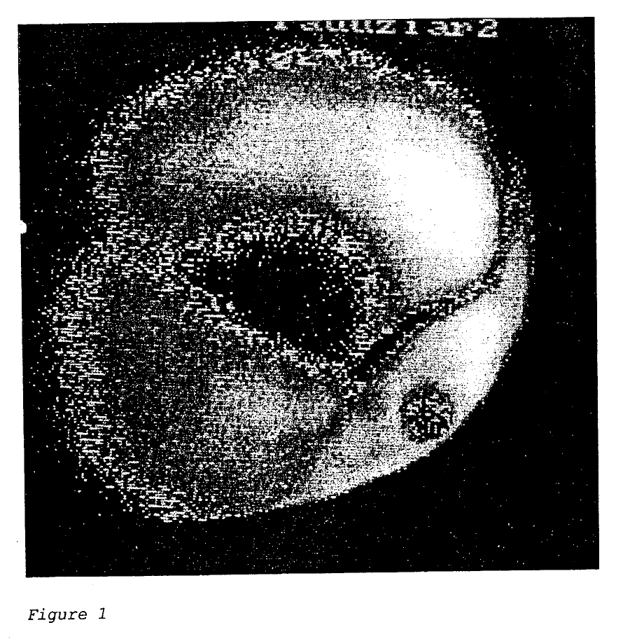

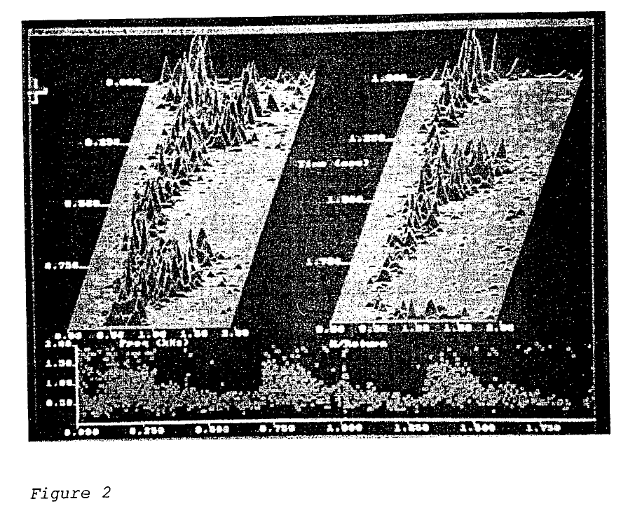

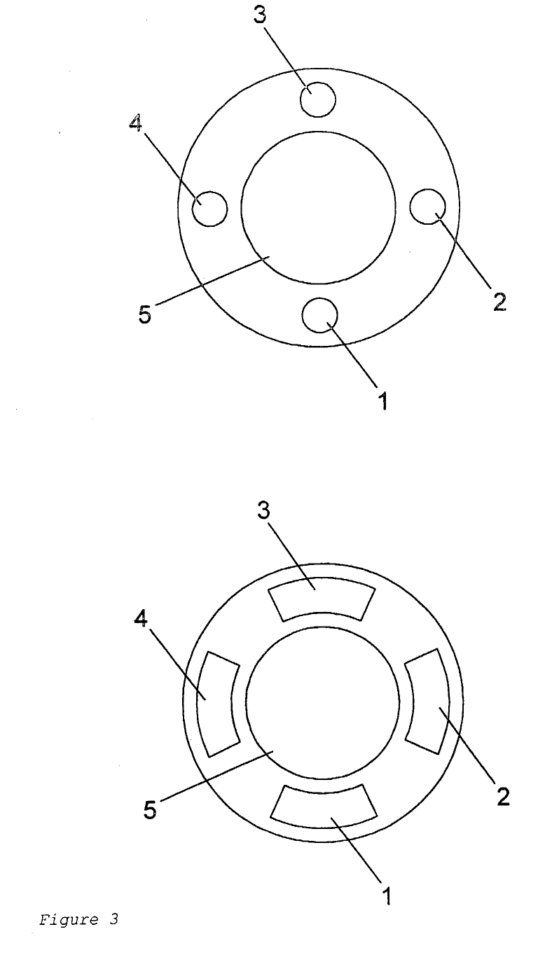

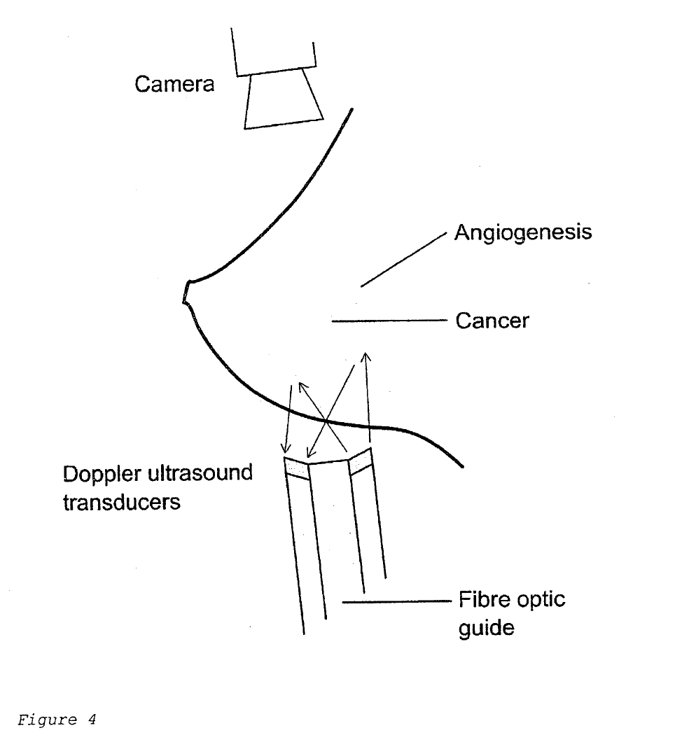

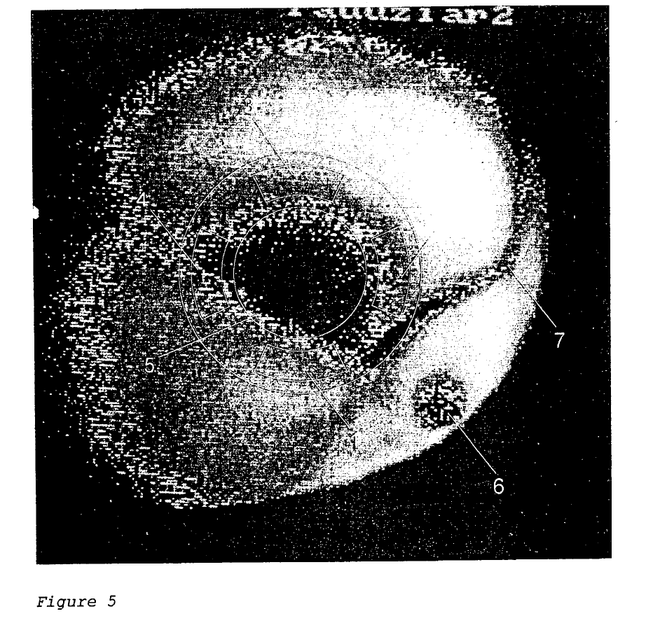

The invention is described by way of example. FIG. 1 shows an image obtained by transillumination recorded by a high sensitivity [moonlight] video camera as described in U.S. Pat. No. 5,007,428 Watmough. The large dark area centrally situated is due to light absorption by angiogenesis surrounding a cancer about 2 cm deep in breast tissue. The nipple [small round dark area] and the superficial blood vessel [black line] on the right of the picture are typical features of optically produced images. FIG. 2 is an isometric display of Doppler frequency shift signals from the angiogenesis around an advanced cancer. The characteristic features of malignancy described above are present. FIG. 3(a) is a sectional drawing of a light guide [5] surrounded by Doppler ultrasound transducers 1, 2, 3 and 4. The combined optical Doppler ultrasound probe is normally placed on the underside of the breast and the light intensity, arranged to be incremented in steps, is varied until an image such as that in FIG. 1 is obtained. The circular disc shaped piezoelectric Doppler transducers are activated so that [1] and [2] transmit and [3] and [4] receive. The difference frequency f_{D} measures the the blood flow in a large fraction of the advancing front of the tumour so that inhomogeneities in neovascularisation are unimportant. FIG. 3(b) shows how a biggger fraction of tumour surface can be interrogated by changing the shape of the Doppler transducers. The physical combination of the optical and ultrasound Doppler techniques in a single probe enable serial examinations to be carried out on a patient with the transducers replaced at the same site on each occasion. Where this condition is not met the ultrasound frequency shift spectra will not in general be comparable and no conclusions about the effect of antiangiogenic drugs can be drawn. FIG. 4 shows the arrangement in vertical section. Note the angulation of the Doppler transducers towards the tumour and the arrows indicating reflection of ultrasound waves from the chaotic mass of vessels onto the receiving transducer(s). Although four transducers have been described any even number can be used. The inputs and outputs can be electronically processed in pairs or in parallel arrays. The preferred operating frequencies are 6, 8, 10 and 12 MHz and there may be several different probes associated with each instrument or in a single probe there can be opposite pairs of piezoelectric elements chosen to operate at different frequencies i.e. one pair at 8 MHz and another at 12 MHz. The reason for the choice of different frequencies is that the tumour depth may vary from patient to patient and 8 MHz may be optimum in one case and 12 MHz in another. Whatever frequency is chosen must be maintained for each of the serial examinations unless two frequencies are chosen for each examination. FIG. 5 shows by superposition how the light guide and the transducers are arranged with respect to the cancer. Only when the light is directed from the same position and at the same intensity will comparable images be obtained. The light intensity must therefore be measured and the numerical value must be stored and shown on the same monitor as the image. New examinations will start by displaying the previous image and varying the probe position until the geometry of a new image resembles as closely as possible the former one. A display where the former and present image are seen side by side is preferred. Landmarks such as nipple and supercial vessels not associated with angiogenesis may be used to locate the probe. In both images the intensity of illumination must have been maintained constant. Differences, such as the size of the dark area in FIG. [1], will then correspond to the effect of drugs on the angiogenesis. Disappearance of blood vessels comprising the angiogenesis will cause the dark area to diminish or vanish altogether. The above procedures will ensure that the Doppler ultrasound examination produces changes in spectra which can be interpreted in terms of the effect of the drugs. Details of signal processing using a computer or dedicated microprocessor are known in the established art. See for instance U.S. Pat. No. 5,007,428 Watmough and Wells PNT [1978] Biomedical Ultrasonics, Academic Press. What has not been taught by the prior art is how to monitor a cancer [breast or testicular] by combined optical and Doppler ultrasound means in such a way that the tests reveal the effect of antiangiogenic and other chemotherapeutic drugs over a protracted period or indefinitely.

References

[1] Watmough D. J. [1983]. Diaphanography. Chapter 6 in the book Medical Imaging [Editor Daphne Jackson ] Surrey University Press, 217-225.

[2] Watmough D. J. [1982]. Diaphanography; Mechanism responsible for the images. Acta. Radiologica Oncol. 21, 11-15.

[4] Watmough D. J., Quan K. M., Aspden R. M., and Mallard J. R. [1992] Phantom study of tissue compression: possible implications for the use of X- ray mammography as a method of imaging breast carcinoma. Europ. J. Surg. Oncol. 18, 538-544.

[5] Watmough D J, Bhargava S, Memon A, Roy S, Syed F [1997] Does breast cancer screening depend on a wobbly hypothesis? Journal Public Health Medicine.19 No. 4, 375-379.

1. An apparatus for examining a body of living tissue containing a neoplasm comprising a light source for illuminating the tissues and means for receiving the radiation transmitted through the tissues, means for producing an image of the said radiation, means for applying ultrasound to the region incorporated in the optical applicator, means for receiving reflected ultrasound from the advancing front of the cancer, characterised by means for producing a signal which is representative of the velocity of blood flow around the tumour and means for generating from the signal a graphical representation which provides an indication of angiogenesis and associated blood flow.

2. An apparatus according to claim 1 wherein means for applying ultrasound and means for receiving reflected ultrasonic signals comprises an ultrasonic Doppler bloodflow detector whose piezoelectric transmitting and receiving elements are incorporated in the housing of the light guide.

3. An apparatus according to claim 1 or claim 2 further comprising a switching device to which the said means for receiving electromagnetic radiation and said means of receiving reflected ultrasound signals are connected and a computer which is connected to the switching device, the switching device being selectively switchable between the said two receiving means thereby to select which of the said two receiving means is connected to the computer.

4. The combined optical ultrasound Doppler probe according two claim 1, claim 2 and claim 3 which has n piezoelectric elements where n=2, 4, 6, 8, 10, 12, and said transducers may be operated in pairs [diagonally opposite or adjacent pairs] so as to obtain ultrasound Doppler signals from a hemispherical tissue volume around the cancer being monitored.

5. An apparatus according to claim 1, claim 2, claim 3 and claim 4 where the geometry of the piezoelements is in the form of annular segments or a series of circular discs all of which may in the plane at right angles to the optical axis of the light guide or inclined to that plane at angles up to about 45 degrees.

6. An apparatus according to claims 1, claim 2, claim 3, claim 4 and claim 5 but where alternate pairs operate at 8 and 10 MHz or 10 MHz and 12 MHz or other similar frequency combinations.

7. An apparatus according to claims 1 to 6 where the optical radiation is incremented in steps so that serial examinations lead to images of tissues and tumour comparison of which monitor the effect of antiangiogenic drugs on neovascularisation on the cancer.

8. An apparatus according to claims 1 to 7 where comparison of Doppler frequency shift spectra from serial examinations reveals the time course of action of antiangiogenic drugs on a cancer in the breast of patients.

9. An apparatus as described in the claims 1 to 8 but adapted to the examination of testicular tissues.

10. Apparatus according to claims 1 to 9 but where contrast media are injected into the vascular system to increase sensitivity of Doppler ultrasound and optical interrogation of tissues.
